(19) United States
(12) Patent Application Publication
(10) Pub. No.: US 2007/0167922 A1
(43) Pub. Date: Jul. 19, 2007

(54) Cannula components

(75) Inventor: Prakash P. Punjabi, London (GB)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(73) Assignee: Hammersmith Hospital's NHS Trust, London (GB)

(21) Appl. No.: 11/569,310

(22) PCT Filed: Jun. 16, 2005
(86) PCT No.: PCT/GB05/02379

§ 371 Date: Feb. 15, 2007

(30) Foreign Application Priority Data
Jun. 17, 2004 (GB) 0413668.5

(51) Int. Cl. A61M 25/00; A61M 5/00

(52) U.S. Cl. 604/264

(57) ABSTRACT

A set of cannula components, the cannula being of a kind which has an end region of sufficient dimensions as to be located, in use, in an aperture of body tissue, a thread being woven around the aperture so that, when the thread is drawn, the fluid is primarily channelled through the cannula, wherein the set comprises at least one clamp configured to tightly fasten onto the outside of the wall of the cannula whilst in a region being sufficiently spiced from the cannla wall to be able to accommodate the passage of a flexible wall tube through which a thread may be drawn; and at least a second clamp configured to squash said flexible wall tube in order to substantially immobilise a thread located inside the tube in order to keep the thread in its desired position.

### FIELD OF THE INVENTION

The invention relates to cannula components. The cannula is of a kind (hereinafter referred to as a cannula of the kind in question) which has an end region of sufficient dimensions as to be located, in use, in an aperture of body tissue, a thread being woven around the aperture so that, when the thread is drawn, the fluid is primarily channelled through the cannula.

### BACKGROUND TO THE INVENTION AND PRIOR ARTR KNOWN TO THE APPLICANTS

Prior art documents have been identified which show a variety of cannula components destined to ease the manner in which purse string threads are secured in tension. Any simplification of operation will be particularly beneficial in this field where reliability is essential and time is literally vital.

FR2628964 shows an arrangement which can be clipped to a cannula for clamping between a surface of an eccentric and a fixed inner surface of the arrangement the threads of a purse string. The end of the rotation of the eccentric causes a spring to be released to apply extra tension onto the thread.

EP1021989A teaches the use of a cannula with tubes mounted on opposite sides of the cannula body. The cannula body and the tubes are either a single body or simply held together by adhesive. A button is mounted radially outwards from the end portion of the tube to allow a surgeon to wrap the thread around the button for a sufficient number of revolutions until the thread is frictionally retained thereon.

U.S. Pat. No. 4,535,770 teaches the use of a surgical steel cannula for conveying blood to or from a blood vessel. A flexible tube may be clipped onto the cannula by a number of clips. Integral with the tube is a cap which, when inserted into the top of the tube, traps the threads between the cap and the inner surface of the tube to hold them in position.

The cannula components shown in these prior art documents are either a complex assembly of parts or directly integrated as part of a cannula tube, thus difficult to operate and prohibit retrofitting to existing cannulas.

### SUMMARY OF THE INVENTION

In a first broad independent aspect, the invention provides a set of cannula components, the cannula being of a kind (hereinafter referred to as a cannula of the kind in question) which has an end region of sufficient dimensions as to be located, in use, in an aperture of body tissue, a thread being woven around the aperture so that, when the thread is drawn, the fluid is primarily channelled through the cannula, wherein the set comprises at least one clamp configured to tightly fasten onto the outside of the wall of the cannula whilst in a region being sufficiently spaced from the cannula wall to be able to accommodate the passage of a flexible wall tube through which a thread may be drawn; and at least a second clamp configured to squash said flexible wall tube in order to substantially immobilise a thread located inside the tube in order to keep the thread in its desired position.

In a second broad independent aspect, the invention provides a cannula component suitable for use on a cannula of the kind in question, configured to tightly fasten onto the outside of the wall of the cannula whilst being in a region sufficiently spaced from the cannula wall to be able to accommodate the passage of a tube through which a thread may be drawn. This configuration is particularly advantageous because it can be used to precisely locate the flexible tubes relative to the cannula for optimal clamping of any threads located in the flexible tube.

In a third broad independent aspect, the invention provides a cannula component suitable for use on a cannula of the kind in question, comprising: a jaw with two halves separated by a hinge, the jaw being able, when shut, to flatten a flexible tube in order to immobilise a thread located inside the tube; wherein the jaw incorporates an eye in the hinge to allow an operator to thread the flexible tube into the jaw through its hinge.

In a fourth broad independent aspect, the invention provides a cannula component, the cannula being of the kind in question, wherein the component comprises means for securing the component on such a cannula and means configured to clamp both a flexible tube and said thread which function separately from said securing means.

This configuration is particularly advantageous because it allows a surgeon to use the flexible tubes which he has been accustomed to use and retain their benefits whilst not having to clamp the purse string threads in the tube by using a separate thread as is often practised in today's operating theatres. This configuration improves the efficiency of the mounting of the cannula to a particular body tissue.

In a fifth broad independent aspect, the invention provides that the component is a collar incorporating a hinged projection which clips onto the collar's body and serves to hold the thread in tension.

This configuration is particularly advantageous because it may be used with standard cannulas. It allows efficient securing of both the collar to a cannula and the thread in tension to the collar.

In a subsidiary aspect in accordance with the invention's fifth broadest aspect, the collar incorporates first and second hinged projections which clip onto two separate regions, whereby threads may be diametrically oppositely held. This collar will allow the advantageous holding of two sets of threads. The provision of these hinged portions will locate the point at which the threads are clamped which will reduce the complexity of operation.

In a further subsidiary aspect, the collar has opening ends which overlap one another and are configured to allow them to be clipped together. In this optional configuration the actions of securing the collar to a cannula and the thread to a collar are carried out by similar operations which simplify the mounting process.

In a sixth independent aspect, the invention provides a cannula of the kind in question, comprising means for holding a thread in tension, characterised in that the means for holding a thread in tension incorporate a hinged projection which clips onto the cannula. One of the advantages of this structure is that it is straightforward to operate.

In a seventh broad independent aspect, the invention provides a cannula component, the cannula being of the kind in question, the component comprising a tube into which threads may be located, characterised in that the tube is formed from a succession of thread locating portions spaced one relative to another and equipped with openings allowing an operator to laterally place the thread into a succession of said portions. This configuration allows an operator to readily place threads in the tube without having to longitudinally thread. This configuration may also simplify the surgeon's required actions.

### BRIEF DESCRIPTION OF THE FIGURES

FIG. 1 shows, in perspective view, a cannula component mounted on a cannula with a tube formed from a succession of thread locating portions.

FIG. 2a shows two collars mounted on a cannula in perspective view.

FIG. 2b shows an end view of a collar in one mode of operation, whilst FIG. 2c shows a second type of collar in a second mode of operation.

FIG. 3a shows a third type of collar mounted on the end of a cannula and in perspective view.

FIG. 3b shows the use of the collar of FIG. 3a simultaneously with two clamps. The view of FIG. 3 is a perspective view.

FIG. 4 shows a further cannula arrangement.

FIG. 5 shows a further type of clamp in perspective view.

FIG. 6 shows an outer perspective view of a flexible tube cannula component.

FIG. 7 shows an inner perspective view of the cannula clamp component of the embodiment of FIG. 6.

FIG. 8 shows a first perspective view of the component in its shut configuration.

FIG. 9 shows a second perspective view of the component in its shut configuration.

FIGS. 10a and 10b show a perspective view of a cannula clamp and a side view of a cannula component respectively in accordance with a further embodiment of the invention.

### DETAILED DESCRIPTION OF THE FIGURES

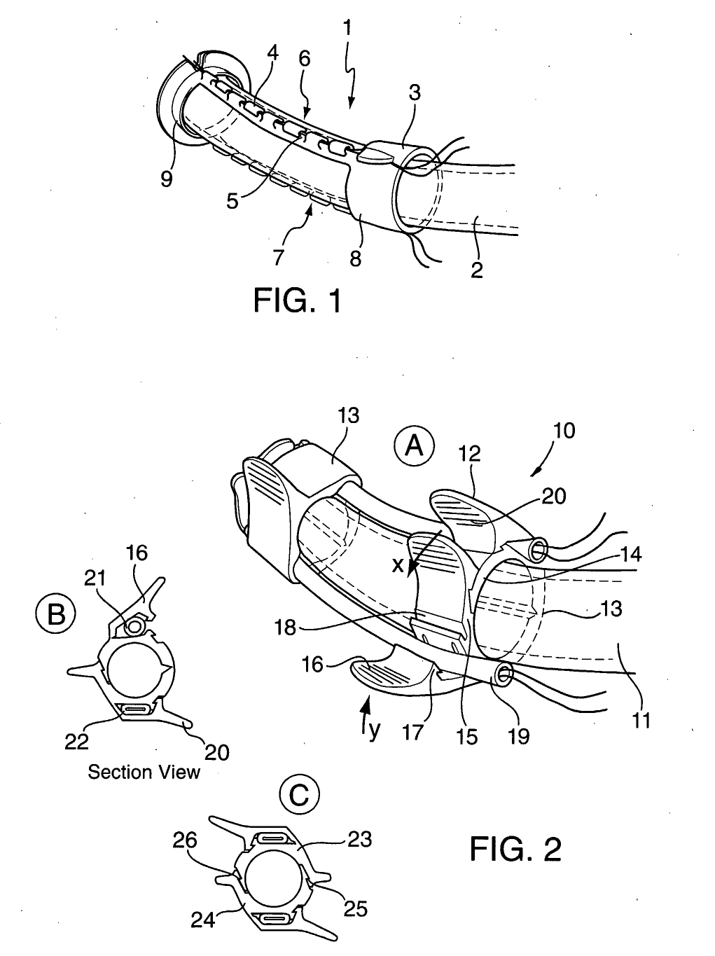

FIG. 1 presents a cannula arrangement generally referenced 1 with a cannula 2 of known kind onto which is mounted a cannula component 3. Component 3 incorporates a succession of thread locating portions such as that referenced 4 running in the longitudinal direction of the cannula 2. Openings such as that referenced 5 allow a user to laterally place the thread into a given thread locating portion of the tube. The cannula component comprises diametrically oppositely located tubes 6 and 7 for individually guiding a thread. A ring 8 and a ring 9 extend around cannula 2. No material is provided between the rings and the tubes.

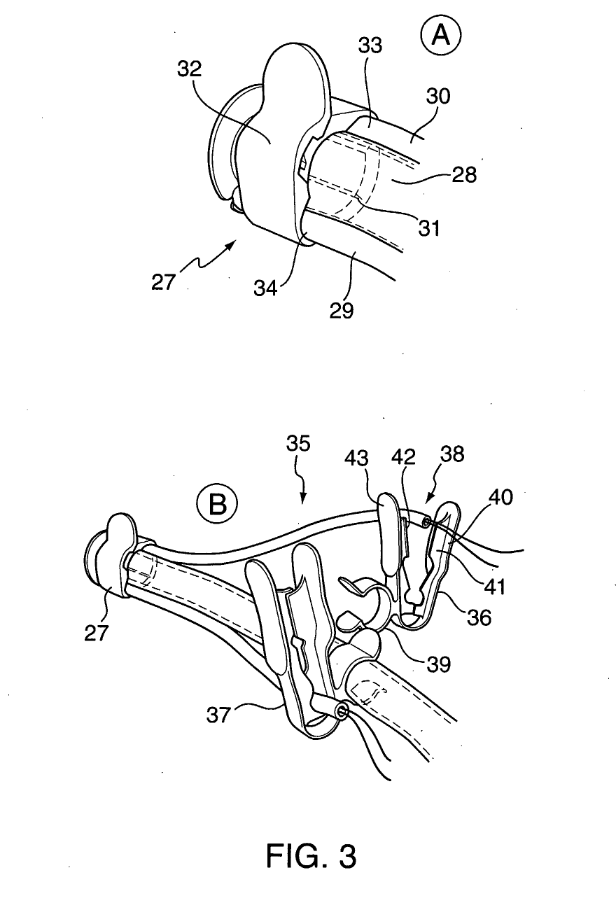

FIG. 2a shows a cannula arrangement generally referenced 10 with a cannula 11 onto which are mounted a collar 12 and a collar 13. Collar 13 will be described in detail with reference to FIG. 3a.

Collar 12 incorporates a hinge 13 extending in the longitudinal direction. It also incorporates two ends 14 and 15 which overlap sufficiently to be clipped together. By applying a force in the direction X (as shown in the figure) the collar may be opened whist hinge 13 allows both ends to be readily separated sufficiently to allow an operator to remove the collar from the cannula.

Projection 16 is equipped with a male member 17 which may be clipped into the female member 18 for securing projection 16 shut. Should a flexible tube such as that shown at reference 19 be located within the jaws of this clamp and should the projection be shut, the flexible tube would be squashed and the threads clamped into position.

A similar projection to projection 16 is provided and referenced 20. This projection may similarly clip to the body of the collar and allow the squashing of both flexible tube and the threads to immobilise them.

FIG. 2b shows projection 16 which does not squash flexible tube 21 whilst projection 20 is clipped shut and squashes flexible tube 22.

FIG. 2c shows a collar formed from two separate body portions 23 and 24 which are clipped together at end regions 25 and 26. Each body portion amounts substantially to half the circumference of the collar.

FIG. 3a shows a collar 27 mounted on a cannula 28 around two tubes 29 and 30. The collar has a hinge 31 and a clip portion 32. The collar is spaced from the cannula at portions 33 and 34 to accommodate the passage of tubes 29 and 30 without squashing the tubes.

FIG. 3b shows a cannula arrangement generally referenced 35 using a collar 27 and two clamps 36 and 37. Clamps 36 and 37 have a jaw portion such as that referenced 38 and a three quarters of a ring portion 39 sufficiently resilient to clip the clamp onto the cannula. The jaw portion 40 incorporates a projection 41 which projects inwardly and which is so configured to correspond to similar projection 42 of opposite jaw portion 43 so that jaw portion 40 and jaw portion 43 may be clipped together. Lower regions of the projections 41 and 42 are set to overlap without clipping together. Thus when flexible tube is located in the jaw and the jaw is clipped shut, the flexible tube is squashed and any thread housed in the tube is immobilised.

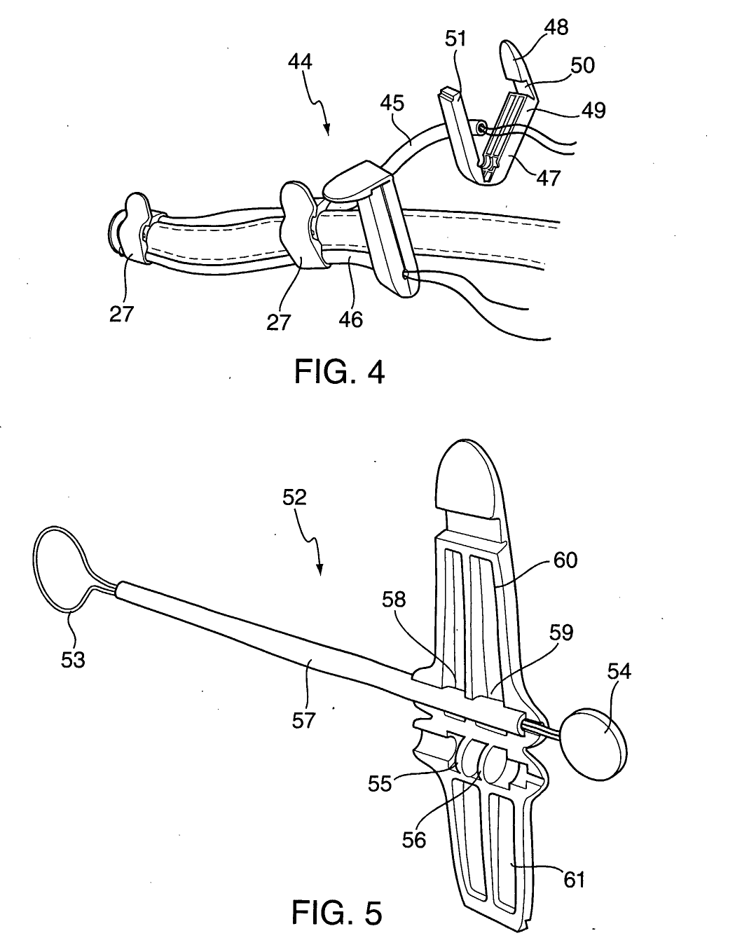

FIG. 4 shows a further cannula arrangement generally referenced 44 which uses two collars of the kind referenced 27 in the previous figures. One of these collars is located at one extremity of the cannula whilst the other is located a distance from the first collar, sufficient to allow an operator to readily clamp the ends of tubes 45 and 46 by using a clamp such as that referenced 47. Clamp 47 incorporates projection 48 extending perpendicularly to jaw portion 49 having a recessed portion 50 configured so that jaw portion 51 may be clipped into projection 48.

FIG. 5 shows a tube and clamp cannula assembly generally referenced 52. In this configuration, the tube may be co-moulded or bonded to the clamp. A thread 53 may be located within the tube whose ends are attached to a button 54. The clamp incorporates two disc shaped projections 55 and 56 which would cause a flexible tube 57 to deflect within recesses 58 and 59 in order to squash thread 53 when sides 60 and 61 of the arrangement are folded together.

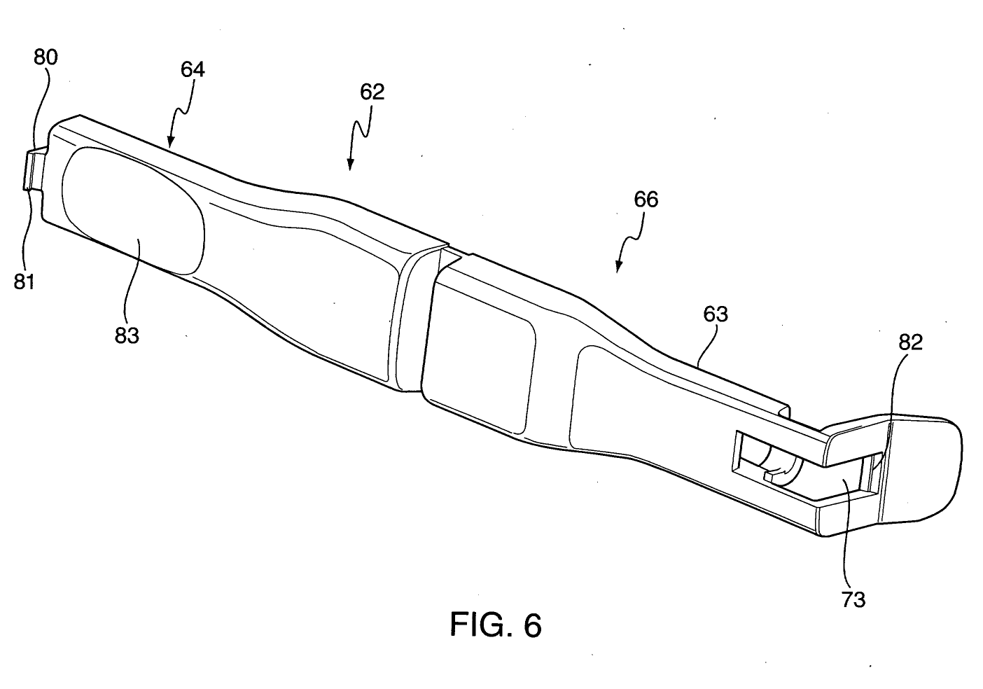

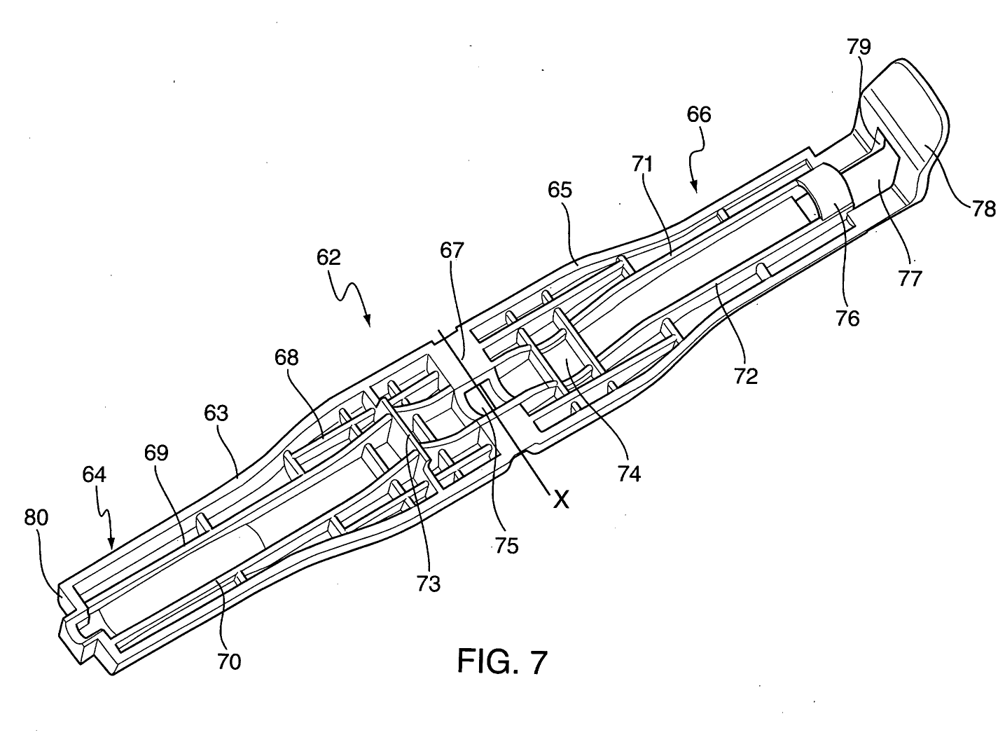

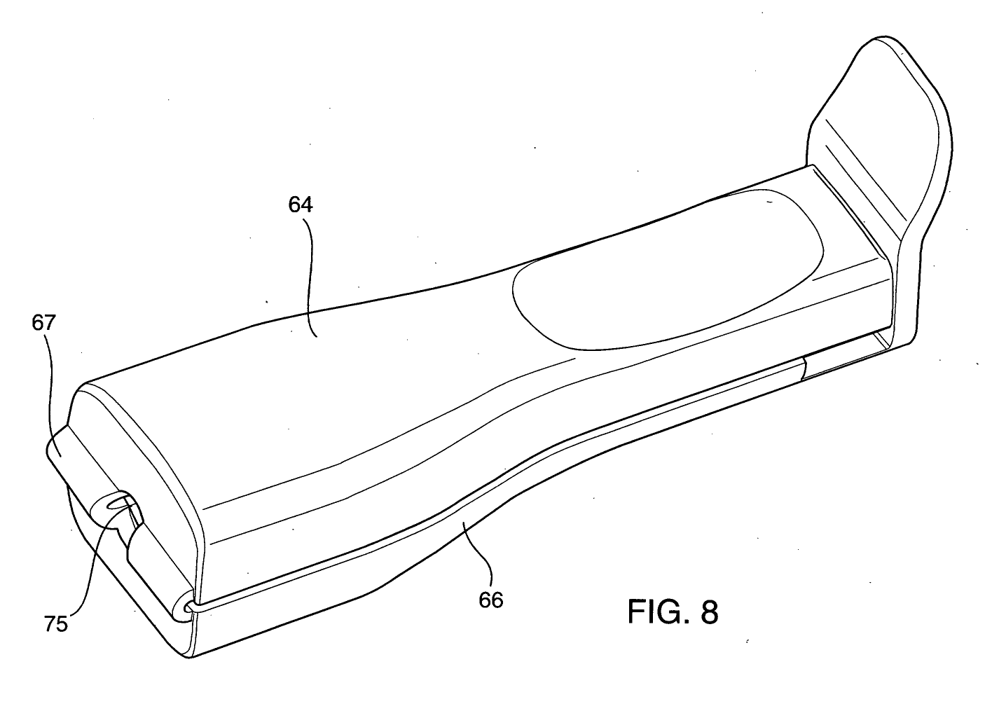

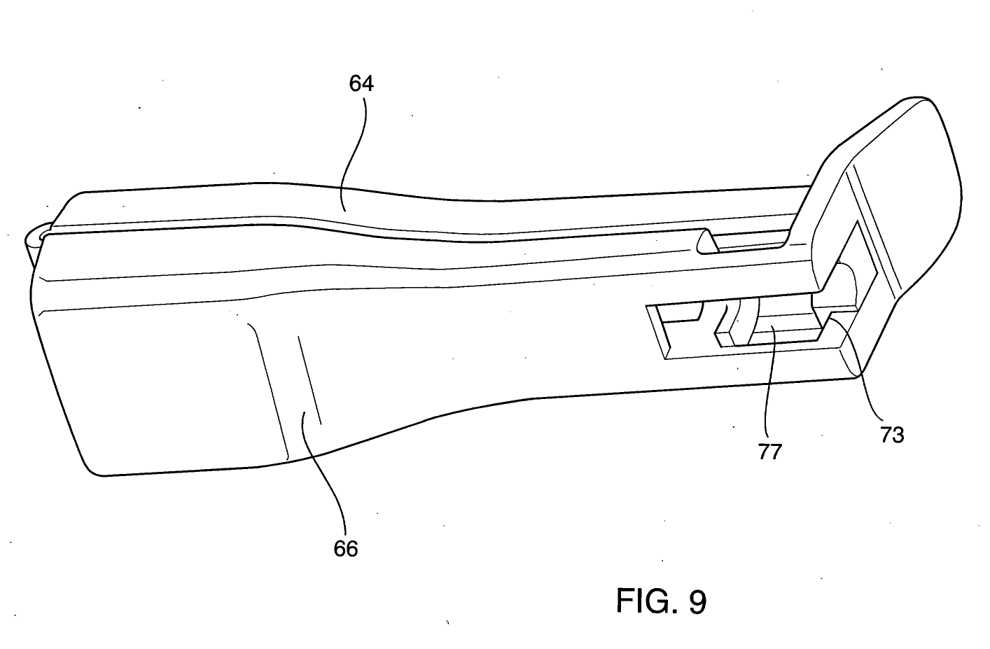

FIG. 6 shows a cannula component 62 used to clamp a flexible tube and the thread which would be contained in the tube. FIG. 6 shows a perspective view of the outside of the cannula component 62 with its jaw opened flat. FIG. 7 shows cannula component 62 with its jaw opened flat. The view shown in FIG. 7 is an inside view. The cannula component or clamp comprises an outer shell 63 of a first half 64 and a second outer shell 65 of a second half 66. Halves 64 and 66 fold together about axis X to close the form shown in FIGS. 8 and 9. In order to allow the opening and closing of the clamp 62, a flexible hinge 67 is provided along axis X. Each half is funnel-shaped with a wider portion in the region adjacent to the hinge 67 and a narrower portion extending from said wide portion. Each shell comprises internal webbing strips such as the strip referenced 68 which reinforce the shell. Strips 69 and 70 extend in the longitudinal direction of the clamp and are spaced in order to be able to receive a flexible tube. Similarly, in half 66, there are provided strips 71 and 72 which also define a space to receive the flexible tube. In the wide portion of funnel-shaped half 64, strips 69 and 70 slope upwards and then downwards towards hinge 67. At their uppermost points, there is provided strip 73 crossing between strip 69 and 70.

By contrast, strips 71 and 72 slope downwards and then upwards in the widest portion of the funnel-shaped half 66. Recessed region 74 is configured to tightly engage projecting region 73 so that when a flexible tube is located in the jaw of clamp 62, when the clamp is shut on the flexible tube, the flexible tube is flattened and the thread located inside the tube is trapped in position. Eye 75 is located at a central position of hinge 67 to allow the passage of the flexible tube and thread, the flexible tube and thread can then be clamped between projecting region 73 and recessed region 74 whilst the remainder of the tube is placed between strips 69, 70 and 71, 72. The uppermost part of the flexible tube and thread can then be placed underneath bridge 76 which comprises an inner abutment to prevent the tube displacing in the longitudinal direction. The tail of the threads may exit the clamp through window 77. Lever 78 extends from half 66 in an essentially perpendicular direction and comprises a cut-out 79 corresponding in size to projection 80 of half 64. Thereby, when half 64 is folded on half 66 projection 68 initially causes lever 78 to bend outwards and is then trapped in cut-out 79. Similarly, in order to release clamp 62, lever 78 may be bent outwards, thus allowing half 64 and its projection 80 to be returned to their unfolded configuration.

As can be seen in FIG. 6, projecting member 80 and cut-out 79 incorporate a step 81 and 82 to further secure the projection in the cut-out. As part of half 64, a shallow recessed region 83 is provided which is recessed in ergonomic fashion so as to correspond to the geometry of a typical operator's fingers/thumb.

In FIG. 8, halves 64 and 66 are shown in their closed position with the geometry of hinge 67 and eye 75 shown in more detail.

FIG. 9 shows the clamp in its closed configuration with cut-outs 79 and 77 being shown in more detail.

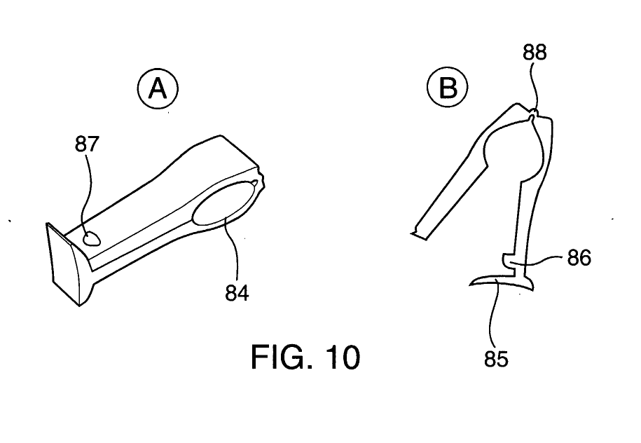

FIGS. 10a and b show a clamp of similar overall geometry to the clamps of FIGS. 6 to 9. These are however configured to clamp around the cannula and two flexible tubes. In order to do so, the wide funnel-shaped region 84 is shaped and configured to clamp around a cannula and two oppositely located flexible tubes. In this configuration, the jaws close across the flexible tubes rather than in their longitudinal directions. Projection 86, lever 85, as well as recess portion 87, are configured to keep the clamp in a closed position whilst at the same time being releasable by tilting lever 85 outwards and prising apart the two clamp halves. Hinge 88 does not comprise an eye to allow the threading of flexible tubes as is the case for the clamps of FIGS. 6 to 9 above.

In order to operate to the satisfaction of a surgeon fitting a cannula during an operation, both types of clamps are employed, the invention therefore envisages these being supplied in a kit of at least one clamp of the kind described in FIG. 10 and at least a clamp of the type described in FIGS. 6 to 9 (preferably two such clamps; one for each flexible tube is envisaged) so that the clamp of FIG. 10 may be used close to the base of the cannula to hold the flexible tubes close to the cannula whilst the clamps of FIGS. 6 to 9 may be used to secure the threads inside the flexible tubes. The latter may usually be carried out by attaching the clamp to the extremities of the flexible tubes, tensioning the threads to an appropriate level and shutting the clamps.

1. A set of cannula components, the cannula being of the kind in question, wherein the set comprises at least one clamp configured to tightly fasten onto the outside of the wall of the cannula whilst in a region being sufficiently spaced from the cannula wall to be able to accommodate the passage of a flexible wall tube through which a thread may be drawn; and at least a second clamp configured to squash said flexible wall tube in order to substantially immobilise a thread located inside the tube in order to keep the thread in its desired position.

2. A cannula component suitable for use on a cannula of the kind in question, configured to tightly fasten onto the outside of the wall of the cannula whilst being in a region sufficiently spaced from the cannula wall to be able to accommodate the passage of a tube through which a thread may be drawn.

3. A cannula component suitable for use on a cannula of the kind in question, comprising: a jaw with two halves separated by a hinge, the jaw being able, when shut, to flatten a flexible tube in order to immobilise a thread located inside the tube; wherein the jaw incorporates an eye in the hinge to allow an operator to thread the flexible tube into the jaw through its hinge.

4-10. (canceled)
